(19) United States
(12) Patent Application Publication
(10) Pub. No.: US 2003/0149436 A1
(43) Pub. Date: Aug. 7, 2003

(54) Fixation and compression fastener assembly for bone fractures

(76) Inventors: Charles L. McDowell, Richmond, VA (US); Winston T. Sanders SR., Chesterfield, VA (US)

Correspondence Address: O'MALLEY AND FIRESTONE, 919 SOUTH HARRISON STREET, SUITE 210, FORT WAYNE, IN, 46802, US

(21) Appl. No.: 10/272,362

(22) Filed: Oct. 16, 2002

Related U.S. Application Data
(60) Provisional application No. 60/354,217, filed on Feb. 4, 2002.

(51) Int. Cl. A61B 17/58

(52) U.S. Cl. 606/73

(57) ABSTRACT

The invention relates to a fastener assembly for repairing a bone fracture having a fastener with a moveable collar and a method for repairing a bone fracture using the fastener assembly to fasten fractured bone pieces together.

### PRIORITY

The present application claims the benefit of U.S. provisional patent application 60/354,217 filed on Feb. 4, 2002.

### BACKGROUND OF THE INVENTION

1. Field of the Invention

The present invention relates to a compression and fixation fastener assembly and a method for repairing bone fractures using the fastener assembly.

2. Description of the Prior Art

Due to trauma such as a car accident, bones can break from impact and stress. Repositioning these bones for healing can be difficult. Broken bones are frequently jagged and uneven. Sometimes the fractured bone fragments displace from each other. Other times the bone fragments jam against each other. Multiple fractures can also occur in a single bone.

Many methods of repairing broken bones treat the bone as an isolated cylinder or rod. For example, the immobilization of a limb or the patient to set the bone treats the bone as if it were a simple cylinder that needs to be glued or affixed together until set. Bone splints, for example, are often used to immobilize bones and joints after fractures or reconstructive surgery. This immobilization can be difficult since the area where the fracture is located may be one that does not readily support casts or is contraindicated for casts.

Broken bone, however, is not a mere broken cylinder in isolation. Bone is living tissue that supports and protects the body. Bone's marrow is rich with blood cells and chondrocytes. Muscles and tendons attach to and move bone. Tendons, however, can pull the healing fractured bone apart during movement which inhibits healing. Because bone also protects organs and nerves, moving bone fragments can also damage the tissue healthy bone protects.

Weight bearing exercise and movement increases bone density, while immobilization decreases bone density. Immobilization can lead to pain and muscle atrophy. Long term immobilization, such as traction, can lead to blood clot formation.

Therefore, for the patient's well-being, methods of repairing broken bones that allow early exercise and movement are desired.

Several methods exist for internally repairing bone fractures. Internal fixation stabilizes the fractured bone by directly fastening the bone pieces to each other internally. Internal fixation of fractures commonly uses plates, rods, screws, wires, nails, and the like, or a combination of thereof. Repairing fractures in large bones, for example, typically involves internal plates screwed into the bone to squeeze or compress the bone fractures together by applying pressure to the exterior of the bone to enhance healing. Plates, however, work poorly with small bones and highly curved bones, such as the bones in the knee joint. Plates also require that the fasteners insert perpendicularly to the exterior of the bone.

Other compression techniques use screws with a modified pitch to screw the bone fragments together. Screwing, however, can damage bone, especially if overtightened or used in fragile bones, such as those in patients with osteoporosis. In addition, screwing the bone fragments together allows spaces to form where the fractures intersect. Screws may also require the screw head to be flush against the bone's exterior, thus reducing a screw's ability to be used on nonplanar surfaces.

Rather than compressing bone fragments together, bone fragments are also fixed together. Fixation with smooth pins such as Steinman pins or wires such as Kirschner or K-wires at the fracture site does not compress the bones together. Threaded pins or wires fixate the bone fragments without compression and are smaller in diameter than smooth pins. Bone fragments are fitted together and pinned, often with multiple pins to prevent the fracture from slipping out of position. Wires are placed through the skin, subcutaneous tissue and bone. Where plaster casts are used, wires or pins can be placed in the cast.

Without a cast or anchor, wires and pins can migrate into the body and cause considerable damage. Wires have sharp ends that can catch on other tissue and other objects, such as clothing. To prevent the sharp ends from catching, surgeons twist the protruding end of the wire or pin. The twisting, however, can weaken and break the wire. The wires may also be difficult to locate for removal if they migrate out of their original position.

If there is subsequent bone loss after fixation of the bone fragments, the pins may not prevent the bone fragments from sliding apart along the pin. This sliding apart or distraction decreases healing. Threaded pins can increase this distraction or the pulling apart of the bone fragments at the fracture site during the first few weeks.

For many of these surgical fasteners, a shaft in the bone fragments must be predrilled before inserting the fastener. Shafts are drilled in the fractured bone pieces and the alignment maintained before inserting the fastener. If the shafts are misaligned, the fastener may not insert within the shaft of the distal bone piece.

Because internal fixation often involves surgery, the patient undergoes a round of anaesthesia and surgery to position and fix the bone fragments together. After healing, the patient may undergo a second round to remove the fastening device, subjecting the patient to additional blood loss and anaesthesia.

Therefore, it is preferable to remove a fastening device without additional surgery. The fastener should be accessible from outside the skin like stitches or near the skin layer where a simple cut under a local anaesthetic can remove the device.

Because the fractured bone must be held together for proper healing, one object of the invention is to prevent the bone from slipping apart when a patient moves. Another object of the invention is to set fractured bones without further damaging the fragile bones and surrounding tissue. A further object of the invention is to both fixate and compress bone using the same device. This device should not significantly interfere with a patient's mobility. Another object of the invention is to insert a device into fractured bone fragments without first drilling a shaft into the fragments.

### SUMMARY OF THE INVENTION

The invention relates to a compression and fixation fastener assembly and a method for repairing bone fractures using the fastener assembly. The fastener assembly has a fastener and a moveable collar. The fastener has a tip, a shank having a shank end distal to the tip, and an intermediate section located between the tip and the shank. The collar has first and second ends as well as a smooth interior wall surrounding the shank. The collar is located between the intermediate portion and the shank end.

The method to repair a bone fracture in a human or animal uses the fastener assembly. The collar is placed onto the shank. First and second bone fragments are positioned together. The fastener tip is aligned on the first bone fragment. A shaft is formed in the first bone fragment. The tip and intermediate section of the fastener are inserted through the first bone fragment and into the second bone fragment. The first and second bone fragments compress together by moving the collar along the shank to a desired position. Next, the collar locks against the shank and keeps the bone fragments together. The bone fragments are then allowed to heal.

Additional effects, features and advantages will be apparent in the written description that follows.

### BRIEF DESCRIPTION OF THE DRAWINGS

FIG. 1 is a perspective view of one embodiment of a fastener assembly of the invention;

FIG. 2 is a perspective view of a collar of the invention;

FIG. 3 is a side view of one embodiment of a fastener assembly of the invention within fractured bone shown in phantom;

FIG. 4 is a side view of a fastener assembly of the invention inserted into a fractured bone shown in phantom;

FIG. 5 is a side view of fastener assemblies of the invention installed within fractured bone shown in phantom;

FIG. 6 is a side view of another embodiment of a fastener assembly of the invention illustrating the movement of a collar down the shaft of the fastener and showing the fractured bone in phantom;

FIG. 7 is a side view of another embodiment of a fastener assembly of the invention;

FIG. 8 is a side view of another embodiment of a fastener assembly of the invention and showing the tissue and fractured bone in phantom;

FIG. 9 is a side view of another embodiment of a fastener assembly of the invention and showing the tissue and fractured bone in phantom;

FIG. 10 is a cross-sectional view of a method of inserting the fastener assembly using an insertion guide and showing the fractured bone in phantom; and

FIG. 11 is a cross-sectional view of another insertion guide for the fastener assembly of the invention.

### DETAILED DESCRIPTION OF THE INVENTION

Turning to the figures where like reference numerals refer to like structures, fastener 10 of the invention has tip 12, shank 14 and intermediate section 16 therebetween. Preferably, fastener 10 is a long, thin rod with a length much longer than its cross-sectional diameter. Fastener can be the diameter of a wire or pin.

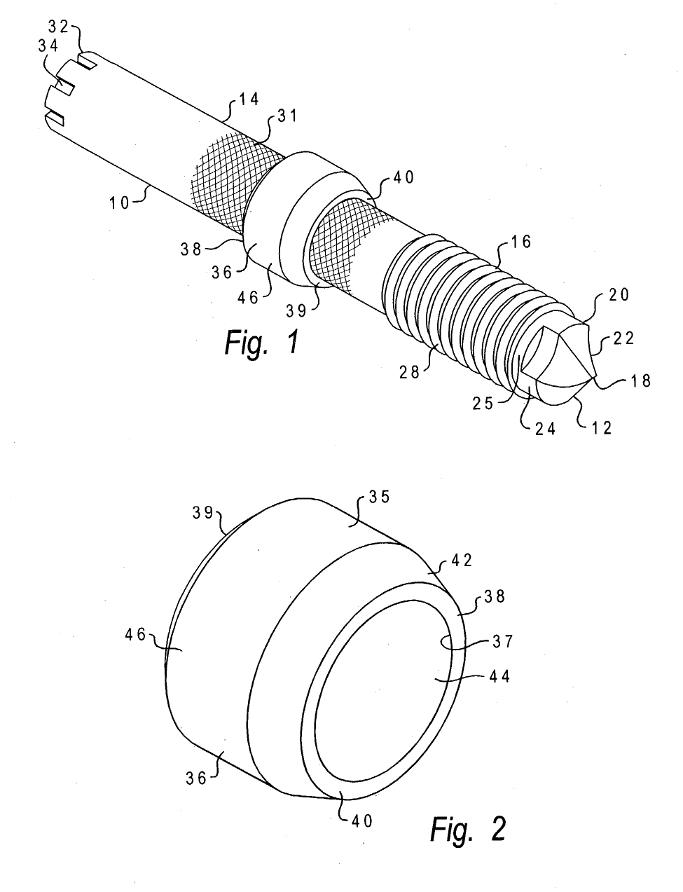

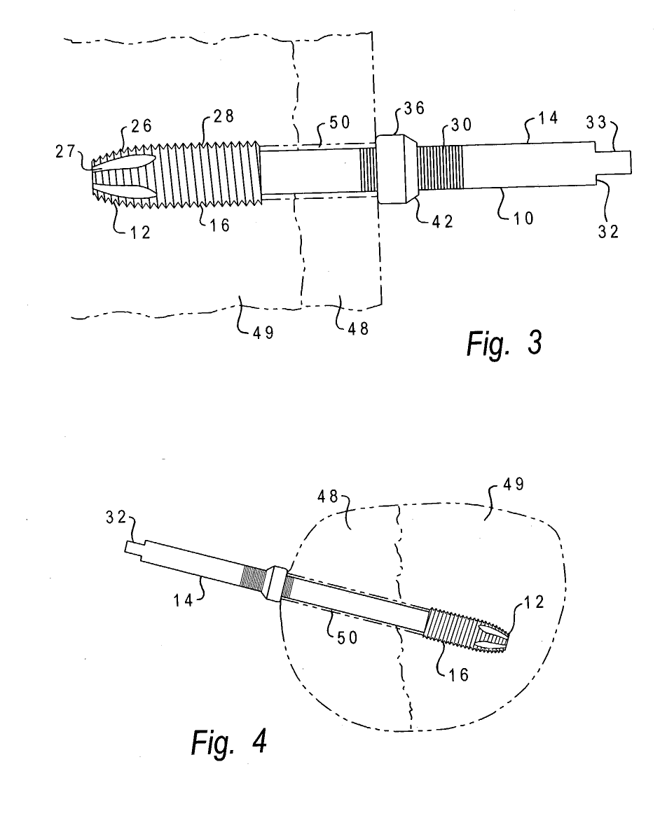

Tip 12 is preferably tapered and cutting with cutting point 18, such as a spade or a trocar tip, and/or blades 20 with grooves 22 located therebetween and terminating in cutting point 18. The tip can also include margin 25 and guiding surface 24 between blades 20 and intermediate section 16 (FIG. 1). Guiding surface 24 helps orient fastener 10 at the desired angle and prevents cutting when at an incorrect angle to minimize damage to the material. Alternatively, tip 12 can be cutting thread 26 with grooves 27, the design of which can also reduce burring during cutting (FIG. 3).

Intermediate section 16 can be textured, such as threaded, ribbed, fluted, and the like, to help pull fastener 10 into the work piece during insertion. Threads 28, for example, are helical.

Shank 14 is longer than intermediate section 16 and tip 12 and comprises more than half of fastener 10. Shank 14 can be textured or smooth, depending on the application. Texturing includes ribs 30 perpendicular to the shank's longitudinal axis, knurling 31, ridges, barbs, hooks, and the like, or a combination of textures at different locations. Shank 14 has shank end 32 which can fit within a chuck or collet of a tool. Such designs can include a flat rectangular end 33 (FIG. 3), teeth, ridges, grooves, and the like, or a combination thereof. Grooves 34, for example, are parallel to the shank's axis which mate with similar ridges on a tool's collet (FIG. 1).

As shown in FIGS. 1 and 3, moveable collar 36 fits onto shank 14. FIG. 2 shows collar 36 having first 38 and second 39 collar ends, and wall 40 having an exterior 35 and an interior 37. Wall 40 defines bore 44. The interior of wall 40 frictionally engages shank 14 and, if desired, bore 44 can be slightly smaller in diameter than shank 14. If desired, wall 40 can be slit from first 38 to second 39 collar ends. Interior 37 can be smooth or textured, depending on the application and shank. For example, interior 37 is preferably smooth if used with a textured shank 14 and textured if used with a smooth shank 14.

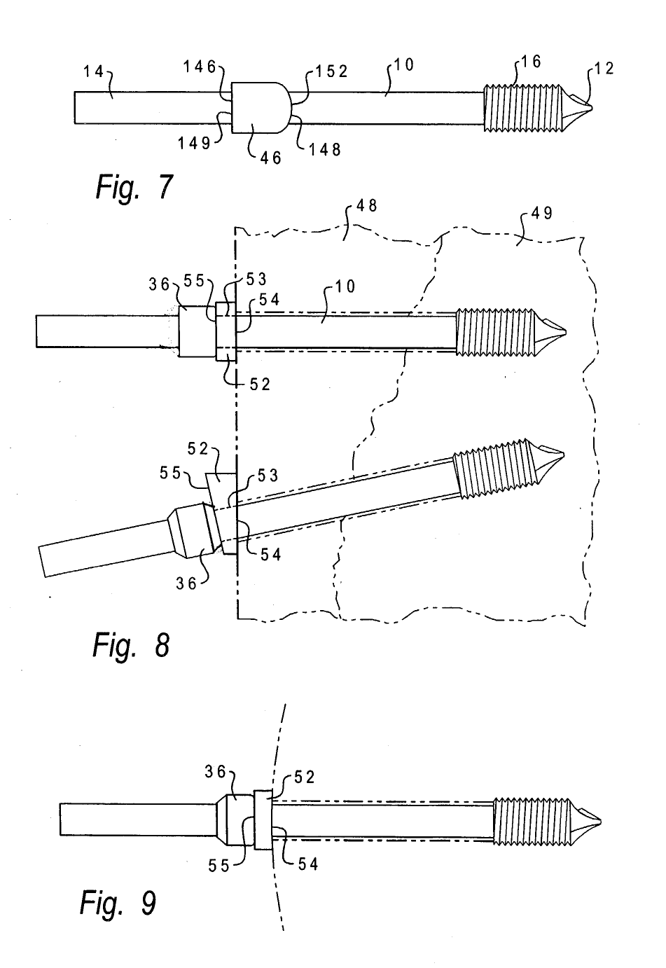

First collar end 38 and second collar end 39 of collar 36 can have different shapes depending on the application. Collar 36, as shown in FIG. 2 for example, can be at least partially frustro-conical, the frustro-conical portion 42 increasing in diameter in cross-section from first collar end 38 until reaching a maximum diameter either at second collar end 39 or at band 46 adjacent to second collar end 39. Bore 44 is preferably of constant diameter from first collar end 38 to second collar end 39. The wall's width, however, preferably varies from first collar end 38 to second collar end 39. Band 46 can be contoured to generate uniform pressure while in use as described below and thus can be of constant diameter or slightly decreasing in diameter in cross-section until reaching second collar end 39. Another alternative is shown in FIG. 7 where collar 146 is at least partially parabolic, with the parabolic portion 152 increasing in diameter in cross-section from one end 148 toward band 46. Band 46 is constant in diameter in cross-section until reaching second end 149. One end can also be concave, if desired (not shown).

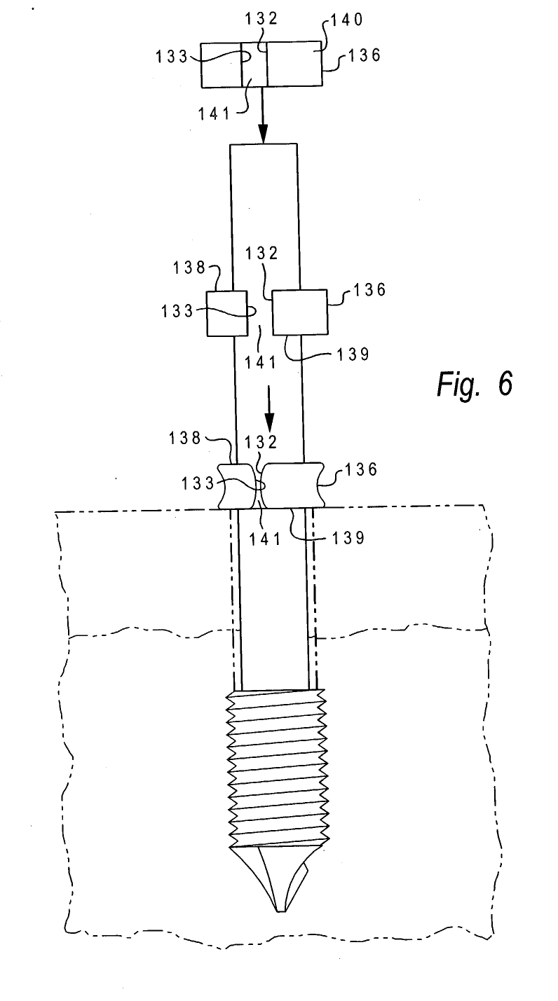

Alternatively as shown in FIG. 6, collar 136 can be of constant diameter in cross-section from first collar end 138 to second collar end 139 and can have slit 141 defined by circumferential wall 140 having first 132 and second 133 ends, if desired. The bore of collar 136 is slightly wider in diameter than shank 14 and therefore does not frictionally engage shank 14. Collar 136 can be crimped, however, which narrows slit 141 and allows collar 136 to frictionally engage shank 14.

In the method of the invention shown in FIGS. 3-5, fastener 10 partially inserts within fractured bone pieces 48, 49. If needed, the fractured bone can first be exposed by a surgical incision at the bone fracture and retracting the tissue away from bone piece 48. Before insertion, tip 12 is aligned in the desired position on the exterior of first bone piece 48. If surgery is unnecessary, tip 12 is placed onto the skin and aligned in the desired position over the location of first bone piece 48 without a surgical incision.

If desired, gasket, ring or washer 52 can be placed between collar 36 and bone piece 48 or skin 41 (FIGS. 8 and 9). Washer 52 has first washer end 54 adjacent bone piece 48 and the second washer end 55 adjacent collar 36 and has a central borehole 53. Washer ends 54, 55 can have different shapes depending on the use, such as a concave first washer end 54 with a flat second washer end 55 (FIG. 9) or a flat first washer end 54 and second washer end 55 angled in comparison to first washer end 54 (FIG. 8). In addition to washer 52 having different shapes, central borehole 53 can be shaped and sized for the application. For example, central borehole 53 is preferably adapted to fit the fastener, which will normally be circular with a diameter wider than shank 14. Central borehole 53 can also be ovoid rather than circular to allow washer 52 to tip or another shape if so desired. Washer 52 can assure that the long, thin fastener is inserted at the correct angle.

During insertion, the texture of intermediate piece 16 should help pull fastener 10 through shaft 50 within bone pieces 48, 49 to the desired depth. In addition, the texture of intermediate section 16 should help anchor or hold fastener 10 within second bone piece 49. The texturing of shank 14 is preferably unidirectional to ease the movement of collar 36 toward bone piece 48 and to prevent collar 36 from moving away from the bone piece 48 during compression.

Shaft 50 can be predrilled into one or both of bone fragments 48, 49 and fastener 10 inserted after drilling. Alternatively, fastener 10 itself can form or enlarge shaft 50 by cutting or drilling into fractured bone pieces 48, 49 during insertion. In this embodiment, shank end 32 of shank 14 is inserted into the chuck of a drill before drilling.

The first position of collar 36 on shank 14 can correspond with the depth of shaft 50. When used in this manner, the collar serves as a stop during insertion after reaching the first bone piece's exterior or the skin.

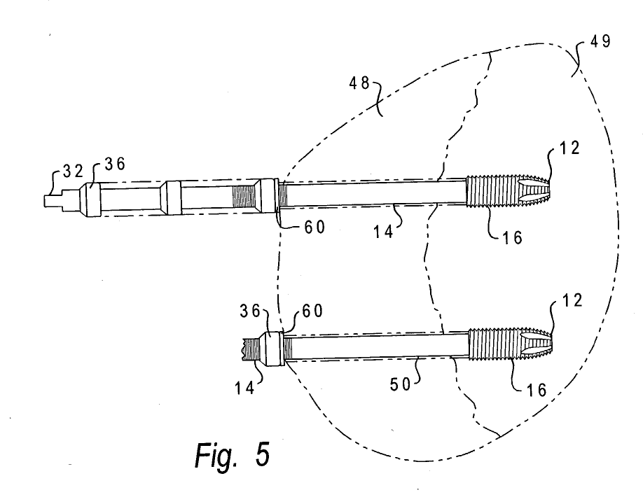

Once fastener 10 is positioned within shaft 50, collar 36 is moved on shank 14 until reaching desired position, such as the exterior of first bone piece 48 or skin. Next, collar 36 is forced against first bone piece 48, skin or washer 52 and locked into position. Any texturing of shank 14 in this position should aid in locking collar 36 in position. Collar 36 can be crimped after moving down shank 14, if desired. After collar placement, shank 14 can be cut at first end 38 of collar 36 or preferably left intact for later removal. For larger bone fractures, additional fasteners can be used (FIG. 5).

FIG. 6 shows fastener 10 positioned within the shaft. Collar 136 slides down shank 14 until reaching the exterior of first bone piece 48. Collar 136 is now preferably crimped to frictionally engage shank 14 to lock fastener 10 in place.

The tight frictional fit of the collar against the shank should prevent the collar from moving on the shank until desired. Because of this fit and the pressure of forcing the collar against the first piece, a tool can be used to provide sufficient external force to ensure adequate compression. Examples of tools include pliers, spreaders, wrenches or compression tools such as a rivet tool adapted to push the collar.

FIGS. 5 and 9 show the fastener of the invention partially inserted within irregularly fractured bone. Collar 36 is positioned on shaft 14 at the desired depth. Fastener 10 is drilled through bone piece 48 and into bone pieces 48, 49 until reaching collar 36. Next, collar 36 is compressed or forced against bone piece 48. After compression, shank 14 is cut or preferably left intact and collar 36 remains locked in position. Alternatively after partially inserting the first fastener but before compression, additional fasteners are similarly partially inserted into the pieces. Next, each collar is positioned in turn against bone piece 48. The ends of the fastener can be capped, if desired. Washer 52 can be placed against bone piece 48, and tip 12 inserted into borehole 53 before drilling fastener 10 into bone pieces 48, 49.

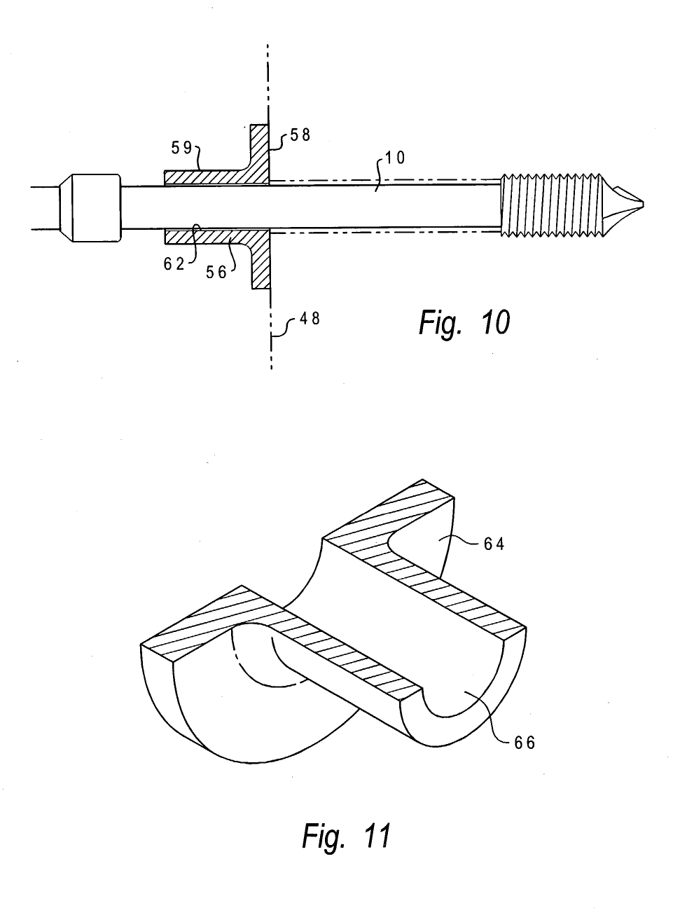

Because of the length of the fastener, an insertion guide such as a jig can maintain the fastener in a perpendicular angle of insertion to the exterior of the bone piece or skin, especially if drilling and inserting are simultaneous. FIG. 10 shows T-shaped insertion guide 56 with flanged end 58 fitting against bone piece 48 and a cylindrical stem 59. Central passageway 62 is adapted to loosely fit fastener 10. Insertion guide 56 can be round. Alternatively, insertion guide 64 has trough 66 onto which fastener 10 rests during insertion (FIG. 11).

Removal of the fastener is simple. If the shank is cut, the collar is cut off and removed or pulled off the cut shaft with a tool, such as a wrench. This will expose a small nub of the cut shaft which can be gripped by a tool, such as a drill chuck, and the fastener rotated until removed from the pieces. Preferably the shank remains intact and the fastener is rotated in the opposite direction to its installation to back it out, such as with a drill chuck attached to the shank end. If the collar or shank are left outside the skin, the fastener is removed without further surgical intervention. Alternatively, if the collar or shank are left just beneath the skin, the fastener is removed with a small incision in the location of the fastener.

The fastener and collar can be made from any material used for surgical fasteners and are well known in the art. The material includes metals, such as surgical steel, titanium, and the like, and organic materials which include plastics with or without fillers, examples of which include polymers and copolyers of medical grade nylon 6/6 and 6, high molecular weight polyethylene, ultrahigh molecular weight polyethylene, polylactic acid, and the like. The collar should frictionally fit against the shank and maintain compression and position on the shank after removing external compressive forces. If a collar is used as shown in FIG. 6, the collar is made of a material that can be crimped to frictionally fit against the shank before moving into position or crimped after placement into position. If desired, the collar can be made from material that is less rigid than the fastener, such as a polymer or copolymer collar with a metal fastener. Washers can also be made of a compressible material, such as silicone, latex and the like.

The fastener and collar can be part of a kit of parts used in an emergency room or hospital. The kit can have fasteners of different diameters that could be used as needed. The kit could also have one or more collars to fit onto the fasteners. A kit with a slit collar 136 for example, may only need one collar as it can be crimped to frictionally fit against shank 14 or expand to fit against a larger shank. A kit with collar 36, however, may require several sizes of collars. Because collar 36 frictionally fits against shank 14, one bore may fit onto fasteners with a variety of diameters ranging in size from one equal to the bore to one smaller than the bore. To accommodate a variety of conditions, the kit may also include collars with differing ends, such as one end being parabolic and the opposite end being flat. A set of washers of different shapes and/or thicknesses could be included in a kit of parts to accommodate a variety of angles of the fastener insertion relative to the surface into which the fastener is inserted. The kit may also include the insertion guide.

The easy to use fastener assembly of the invention has many advantages over other fasteners. The tight frictional fit of the collar against the shank keeps the collar in position. This fixates the bone fragments in position. The collar also maintains compression against the bone to aid in healing. Many previous fasteners require the fastener head to be flush with the first bone piece's exterior, reducing its ability to be used on the nonplanar bone surfaces. The fastener, therefore, can insert at an angle instead of flush with the surface, yet the collar forces the pieces together by compression. Unlike screws, the fastener is not easily overtightened, which decreases the risk of further damaging the bone.

The bone fracture area does not require predrilling before inserting the fastener. If a cutting tip is used, the fastener can cut into the fractured bone pieces to reduce alignment problems.

The fastener assembly is easy to remove after healing. If implanted with the shank end outside the skin, the fastener is simply rotated out of the body.

Another advantage with the invention is that if distraction occurs, the bones can be compressed from outside the body by tightening the collar. Because the fastener is locked into position, the fastener is unlikely to migrate from its location after implantation.

Because the diameter of the fastener is small relative to the size of the bone fragments, the risk of damage to the bone fragments is reduced.

Another advantage of the invention is that multiple fasteners can be used. Multiple fasteners can be inserted in multiple planes for added strength for fixation as compared to single or double plane fixation using screws or plates.

While the invention is shown in only one of its forms, it is not thus limited but is susceptible to various changes and modifications without departing from the spirit and scope of the invention.

What is claimed is:

1. A fastener assembly for repairing a bone fracture comprising:
a fastener comprising a tip, a shank having a shank end distal to the tip, and an intermediate section located between the tip and the shank; and
a moveable collar having first and second ends, and a wall having an exterior and a smooth interior surrounding the shank, the collar being located between the intermediate portion and the shank end.

2. A fastener assembly for repairing a bone fracture of claim 1, wherein the intermediate section is textured.

3. A fastener assembly for repairing a bone fracture of claim 2, wherein the tip further comprises:
cutting means.

4. A fastener assembly for repairing a bone fracture of claim 3, wherein the shank end is adapted to fit within a collet of a tool.

5. A fastener assembly for repairing a bone fracture of claim 3, wherein the shank is at least partially textured.

6. A fastener assembly for repairing a bone fracture of claim 5, wherein the interior of the wall of the collar frictionally engages the shank.

7. A fastener assembly for repairing a bone fracture of claim 6, wherein the collar further comprises:
a slit from the exterior of the wall to the interior of the wall.

8. A fastener assembly for repairing a bone fracture of claim 7, wherein the collar is at least partially frustro-conical and increases in diameter in cross-section from the first collar end.

9. A fastener assembly for repairing a bone fracture of claim 8, wherein the collar further comprises:
a band adjacent to the second collar end.

10. A method of repairing a bone fracture, comprising the steps of:
providing a fastener comprising a tip, a shank having a shank end distal to the tip, and an intermediate section located between the tip and the shank;
placing onto the shank a collar having first and second ends, and a wall having an exterior and a smooth interior defining a bore;
positioning a first bone fragment and a second bone fragment together;
aligning the tip on the first bone fragment;
forming a shaft in the first bone fragment;
inserting the tip and intermediate section through the first bone fragment and into the second bone fragment;
compressing the first bone fragment and second bone fragment together by moving the collar along the shank to a desired position;
locking the collar against the shank;
keeping the bone fragments together after locking the collar; and
allowing the bone fragments to heal.

11. A method of repairing a bone fracture of claim 10, further comprising the step of:
helping pull the fastener into the first bone fragment with the intermediate section; and
wherein the intermediate section is textured.

12. A method of repairing a bone fracture of claim 11, wherein the shank is at least partially textured.

13. A method of repairing a bone fracture of claim 12, wherein the shaft is formed during the insertion of the tip and intermediate section.

14. A method of repairing a bone fracture of claim 13, wherein the collar further comprises a slit from the exterior of the wall to the interior of the wall.

15. A method of repairing a bone fracture of claim 13, further comprising the step of:
crimping the collar against the shank to frictionally engage the shank.

16. A method of repairing a bone fracture of claim 13, further comprising the step of:
frictionally engaging the shank with the interior of the wall.

17. A method of repairing a bone fracture of claim 16, wherein the collar is at least partially frustro-conical and increases in diameter in cross-section from the first collar end.

18. A method of repairing a bone fracture of claim 13, further comprising the step of:
inserting the tip into a central borehole of a washer before inserting the tip into the first bone fragment.

19. A method of repairing a bone fracture of claim 13, further comprising the step of:
guiding the fastener during insertion with an insertion guide.

20. A fastener assembly kit of parts for repairing a bone fracture comprising:
fasteners of different diameters, each fastener comprising a tip having cutting means, a shank having a shank end distal to the tip, and a textured intermediate section located between the tip and the shank; and
a collar having first and second ends, and a wall having an exterior and a smooth interior defining a bore.

21. A fastener assembly kit of parts for repairing a bone fracture of claim 20, wherein the shank is at least partially textured.

22. A fastener assembly kit of parts for repairing a bone fracture of claim 21, wherein the collar further comprises:
a slit from the exterior of the wall to the interior of the wall.

23. A fastener assembly kit of parts for repairing a bone fracture of claim 21, wherein the collar is at least partially frustro-conical and increases in diameter in cross-section from the first collar end.

24. A fastener assembly kit of parts for repairing a bone fracture of claim 21, further comprising:
at least one washer with a central borehole.

25. A fastener assembly kit of parts for repairing a bone fracture of claim 21, further comprising:
washers having opposite washer ends and a central borehole; and
wherein the washers have different shapes.

26. A fastener assembly kit of parts for repairing a bone fracture of claim 21, further comprising:
at least one insertion guide.
